(19) United States
(12) Patent Application Publication
(10) Pub. No.: US 2026/0345201 A1
(43) Pub. Date: Nov. 5, 2026

(54) ANTI-ROLLING AND ANTI-ROCKING TIP COVERS FOR MEDICAL DEVICES AND OTHER TOOLS OR INSTRUMENTS

(71) Applicant: Raine Medical, Inc., Kentwood, MI (US)

(72) Inventor: Gabrielle Kelly, Alto, MI (US)

(21) Appl. No.: 19/650,104

(22) Filed: Apr. 16, 2026

Related U.S. Application Data
(60) Provisional application No. 63/789,954, filed on Apr. 16, 2025.

(51) Int. Cl. A61B 17/34

(52) U.S. Cl. CPC A61B 17/3494

(57) ABSTRACT

Tip covers for medical devices and other tool or instruments are disclosed herein. In some embodiments, a tip cover for covering a distal end of a medical device includes an elongated body extending between a proximal end and a distal end thereof. The elongated body can include (i) a wall having a cross-section shaped generally as an n-degree polygon such that the wall has n flat sides, and (ii) a cavity surrounded by the wall and extending from the proximal end of the elongated body. The cavity can receive the distal end of the medical device therein. When the elongated body is at rest on a surface on any one of the n flat sides, a center of gravity of the elongated body can be positioned no more than 20% of a length of the one of the n flat sides laterally away from a center of the one of the n flat sides.

### CROSS-REFERENCE TO RELATED APPLICATION(S)

This application claims priority to U.S. Provisional Application No. 63/789,954, filed Apr. 16, 2025, which is incorporated herein by reference in its entirety.

### TECHNICAL FIELD

The present technology generally relates to tip covers for medical devices, components, and other tools or instruments, and more particularly to tip covers that resist rolling and rocking on a surface.

### BACKGROUND

Many types of medical devices or components must be sterilized before use. For example, steam sterilization, ethylene oxide (EtO) sterilization, radiation sterilization, and/or other forms of sterilization can be performed to ensure that the medical devices or components are free of any viable microorganisms, including bacteria, viruses, fungi, and spores. Common medical devices or components that may need to be sterilized include scalpels, forceps, scissors, retractors, speculums, and more.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments shown, but are provided for explanation and understanding.

FIG. 1 illustrates various medical devices fitted with tip covers configured in accordance with various embodiments of the present technology.

FIG. 2A-2E are perspective, front, side, top, and bottom views, respectively, of a small-sized tip cover configured in accordance with various embodiments of the present technology.

FIG. 3A-3E are perspective, front, side, top, and bottom views, respectively, of a medium-sized tip cover configured in accordance with various embodiments of the present technology.

FIG. 4A-4E are perspective, front, side, top, and bottom views, respectively, of a large-sized tip cover configured in accordance with various embodiments of the present technology.

FIG. 5 is a schematic plan view of a surgical or medical kit configured in accordance with various embodiments of the present technology.

FIGS. 6A and 6B illustrate the anti-rolling and anti-rocking features of a tip cover configured in accordance with various embodiments of the present technology.

### DETAILED DESCRIPTION

### A. Overview

Embodiments of the present technology are directed to tip covers (also referred to herein as “tip protectors,” “tool covers,” “tool protectors,” or “tip guards”) for medical devices or other tools or instruments. Many types of medical devices, such as scalpels, forceps, scissors, retractors, speculums, and/or the like, may need to be sterilized before use. For example, surgical tools intended to be inserted into an incision on a patient or otherwise contact the patient must be sterilized to ensure that the medical devices are free of any viable microorganisms, including bacteria, viruses, fungi, and spores. These medical devices, however, often include sharp edges, points, tips, and/or the like that may puncture, scratch, break, and/or otherwise damage a sterilization chamber in which the medical devices are sterilized before use. Also, it can be desirable to protect and thereby preserve such sharp edges, points, tips, and/or the like during handling. Therefore, medical devices can be fitted with tip covers before, during, and/or after sterilization.

Existing tip covers for medical devices, however, may be prone to rolling, such as when dropped, placed on a surface, or otherwise moved. For example, a technician may, while unpackaging existing tip covers and/or fitting the existing tip covers on medical devices or other tools, accidentally drop the tip covers on a table or on the floor or may bump a table on which such tip covers are resting. Because such tip covers are prone to rolling, the tip covers can proceed to roll in various directions, including off the table and/or along the floor. This can not only create an inconvenience for the technician having to pick up each tip cover, but can also delay subsequent procedures and may require one or more of the tip covers to be disposed of for sanitary reasons.

Other existing tip covers may be prone to rocking or other movement, such as when placed on a table or other surface. For example, many existing tip covers are shaped such that when such tip covers are fitted onto tips of medical devices and the medical devices are thereafter placed on a table or other surface (e.g., a tray), the tip covers allow the medical devices to move/rock and are therefore unable to keep the medical devices stable on the table or surface. Depending on the medical devices, movement of the table (e.g., due to an operator bumping into the table) or other surface (e.g., during transport of the surface/tray) can therefore permit the medical devices to rock, roll, and/shift positions, which may result (a) in the medical devices subsequently falling off the table or other surface and/or (b) in a technician having to reposition/redistribute the medical devices along the table or other surface (e.g., for assembly prior to packaging or sterilization).

Embodiments of the present technology address at least some of the issues described above. For example, embodiments of the present technology include a tip cover for covering a distal end of a medical device. The tip cover can include an elongated body extending between a proximal end and a distal end thereof. The elongated body can include (i) a wall having a cross-section shaped generally as an n-degree polygon such that the wall has n flat sides, and (ii) a cavity surrounded by the wall and extending from the proximal end of the elongated body. The cavity can be configured to receive the distal end of the medical device therein. When the elongated body is at rest on a surface on any one of the n flat sides, a center of gravity of the elongated body can be positioned no more than 20% of a length of the one of the n flat sides laterally away from a center of the one of the n flat sides.

In some embodiments, a medical kit comprises a plurality of tip covers including one or more small-sized tip covers, one or more medium-sized tip covers, and one or more large-sized tip covers. Each of the plurality of tip covers can include (i) a wall having a cross-section shaped generally as an n-degree polygon such that the wall has n flat sides, and (ii) a cavity surrounded by the wall and extending from the proximal end of the elongated body. When the tip cover is at rest on a surface on any one of the n flat sides, a center of gravity of the tip cover can be positioned no more than 20% of a length of the one of the n flat sides laterally away from a center of the one of the n flat sides. The cavities of the one or more small-sized tip covers, the one or more medium-sized tip covers, and the one or more large-sized tip covers can be sized differently to receive differently sized distal ends of medical devices.

Embodiments of the present technology can provide tip covers having multiple flat sides, multiple lines of symmetry, and a center of gravity that altogether provide a high degree of stability. Accordingly, tip covers of the present technology, regardless of the specific shape or size, can resist rolling and rocking motions on a surface to a higher degree compared to many existing tip covers. The anti-rolling and anti-rocking features of the tip covers described herein are expected to provide several advantages. For example, when tip covers of the present technology drop onto a surface (e.g., a table or tray) or the floor (e.g., due to mishandling by a technician, accidental shaking of a table on which the tip covers are placed), the tip covers are expected to prevent or at least resist rolling and thereby quickly come to a stop. Thus, the tip covers are expected to (i) help prevent the tip covers from rolling off of the surface, (ii) help prevent the tip covers from rolling great distances along the floor and therefore from getting lost (e.g., by rolling underneath a cabinet), (iii) provide a convenience factor to personnel by reducing the effort and time it takes to pick the tip covers back up, and more. As another example, the tip covers of the present technology, when fitted onto medical devices, are expected to resist rocking and/or other movement of the medical devices and thereby retain the medical devices in a stable position, such as when a table or other surface on which the medical devices are placed is moved.

In the following description, specific details are set forth to provide a thorough understanding of aspects of the present technology. One skilled in the relevant art will recognize, however, that the systems, devices, and techniques described herein can be practiced without one or more of the specific details set forth herein, or with other methods, components, materials, etc.

Reference throughout this specification to an “example” or an “embodiment” means that a particular feature, structure, or characteristic described in connection with the example or embodiment is included in at least one example or embodiment of the present technology. Thus, use of the phrases “for example,” “as an example,” or “an embodiment” herein are not necessarily all referring to the same example or embodiment and are not necessarily limited to the specific example or embodiment discussed. Furthermore, features, structures, or characteristics of the present technology described herein may be combined in any suitable manner to provide further examples or embodiments of the present technology.

Spatially relative terms (e.g., “beneath,” “below,” “over,” “under,” “above,” “upper,” “top,” “bottom,” “left,” “right,” “center,” “middle,” and the like) may be used herein for ease of description to describe one element's or feature's relationship relative to one or more other elements or features as illustrated in the figures. It will be understood that the spatially relative terms are intended to encompass different orientations of a device or system in use or operation, in addition to the orientation depicted in the figures. For example, if a device or system illustrated in the figures is rotated, turned, or flipped about a horizontal axis, elements or features described as “below” or “beneath” or “under” one or more other elements or features may then be oriented “above” the one or more other elements or features. Thus, the exemplary terms “below” and “under” are non-limiting and can encompass both an orientation of above and below. The device or system may additionally, or alternatively, be otherwise oriented (e.g., rotated ninety degrees about a vertical axis, or at other orientations) than illustrated in the figures, and the spatially relative descriptors used herein are interpreted accordingly. In addition, it will also be understood that when an element is referred to as being “between” two other elements, it can be the only element between the two other elements, or one or more intervening elements may also be present.

The term “medical device” as used herein encompasses a broad range of instruments, apparatuses, tools, and other components that may be used in a medical context. For example, the term “medical device” can include scalpels, syringes, diagnostic equipment, and/or the like that are conventionally classified as medical devices (e.g., by the U.S. Food and Drug Administration (FDA)). As another example, the term “medical device” can also include orthopedic drill bits, surgical saws, dental instruments, and/or the like that may not necessarily be classified as medical devices (e.g., by the FDA). Accordingly, unless explicitly specified or made clear through context, the foregoing description is not limited to use with conventional medical devices, and should be read as directed to tip covers compatible with a wide range of components.

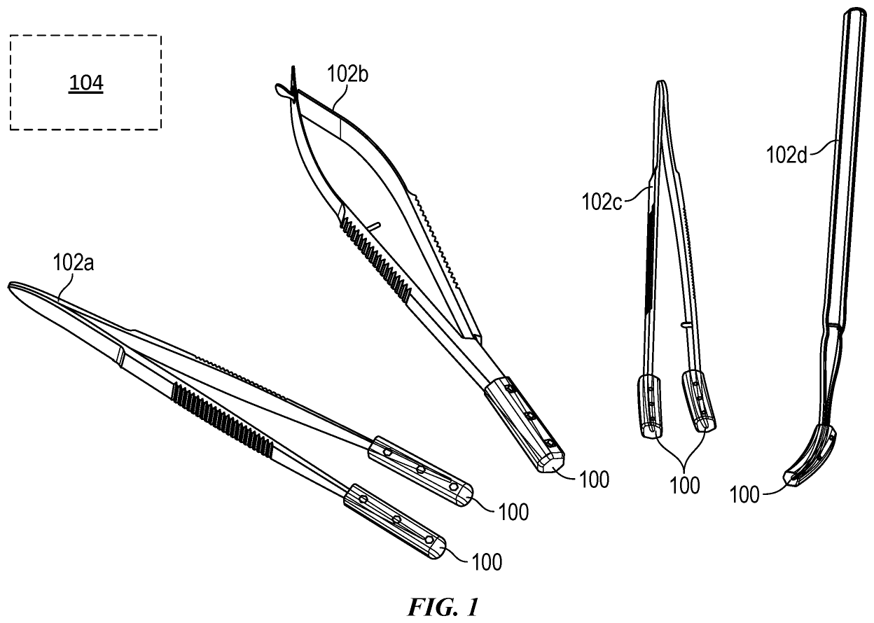

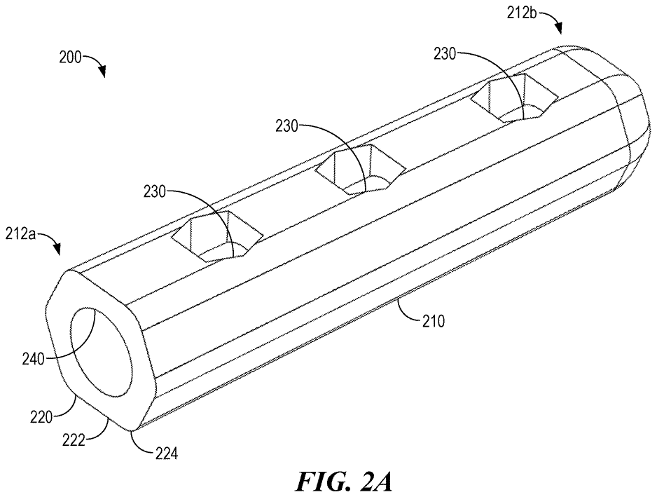

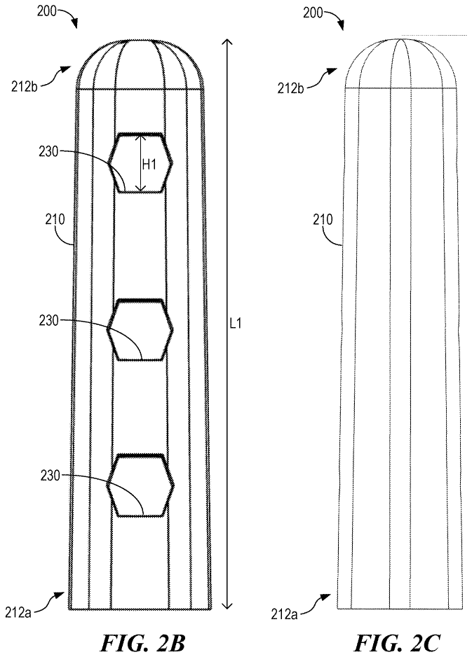

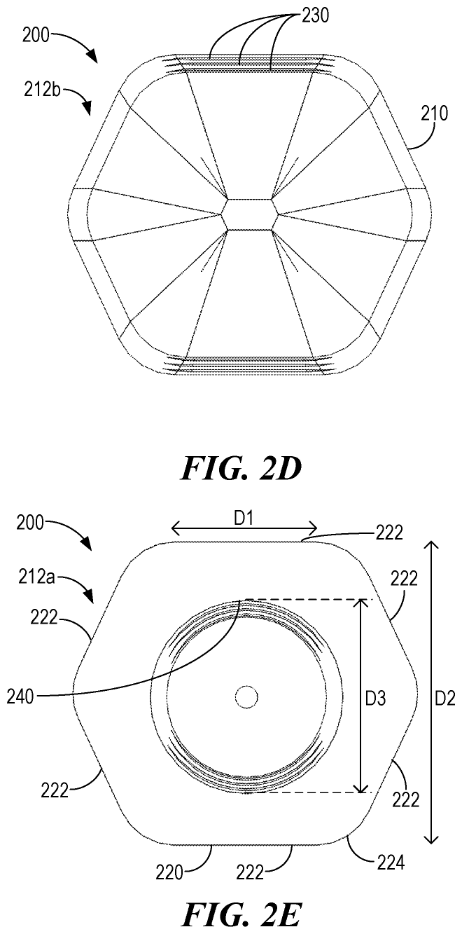

Reference numbers used in the figures of the present disclosure follow a numbering convention in which (i) the first digit or digits correspond to the first figure in which a particular element or component is introduced and (ii) the remaining digits identify that particular element or component in the figures. Unless otherwise specified or made clear from context, similar references numbers are used across multiple figures to denote generally similar and/or identical components. For example, reference number 102 can be used to reference an element “2” that was first introduced in FIG. 1. Use of reference number 102 in FIG. 2 can identify the element “2” from FIG. 1 in FIG. 2. Use of reference number 202 in FIG. 2 can be used to reference an element “2” that was first introduced in FIG. 2, and that may (depending on context) be generally similar and/or identical to the element “2” corresponding to reference number 102 that was first introduced in FIG. 1.

### B. Select Embodiments of Tip Covers and Associated Systems, Devices, and Methods

FIG. 1 illustrates various medical devices 102a-102d fitted with tip covers 100 configured in accordance with various embodiments of the present technology. The illustrated medical devices 102a-102d are merely examples, and other medical devices and/or components can be fitted with the tip covers 100 of the present technology. For example, the tip covers 100 can be used with scalpels, forceps, scissors, retractors, speculums, drill bits, saws, bone reamers, and more. Such medical devices or components can have sharp edges, points, tips, and/or the like at their distal ends.

The tip covers 100, also referred to herein as “tip protectors,” “tool covers,” “tool protectors,” or “tip guards” can be sized and shaped to fit over the distal ends of the medical devices 102a-102d. The tip covers 100 can prevent the sharp ends of the medical devices 102a-102d from directly contacting and thus puncturing, scratching, ripping, or otherwise damaging a sterile barrier pouch 104 (shown schematically and not to scale) in which the medical devices are placed in prior to being sterilized. The tip covers 100 can also prevent the sharp ends of the medical devices 102a-102d from damaging other objects, such as a technician's skin. The tip covers 100 can also protect the distal ends of the medical devices from abrasion, allowing the medical devices to preserve the shapes of their distal ends.

In some embodiments, the tip covers 100 can be made from elastomers (e.g., thermoplastic elastomers (TPE), thermoplastic polyurethane (TPU), silicone), resins (e.g., bio-resins), other non-PVC materials, and/or other suitable materials (including PVC materials). The material can be selected to withstand steam sterilization, ethylene oxide (EtO) sterilization, gamma radiation sterilization, and/or the like. In these and other embodiments, the tip covers 100 can be manufactured via injection molding, additive manufacturing (e.g., 3D printing), and/or other suitable manufacturing techniques.

As discussed in greater detail herein, the tip covers 100 can include shapes and other features that permit the tip covers 100 to resist and/or minimize rolling and/or rocking, such as when placed on a surface. For example, if one of the tip covers 100 is dropped onto or placed on a surface or the floor, features of that tip cover 100 can prevent (or at least resist) rolling and thereby enable the tip protector 100 to quickly come to a stop. By effectively reducing or minimizing the distance by which the tip cover 100 rolls, the tip cover 100 can reduce (i) the likelihood of becoming lost (e.g., by rolling underneath a cabinet), (ii) the effort and time it takes to retrieve and pick up the tip cover 100, and more.

FIG. 2A-2E are perspective, front, side, top, and bottom views, respectively, of a small-sized tip cover 200 configured in accordance with various embodiments of the present technology. The tip cover 200 can be an example of the tip covers 100 illustrated in FIG. 1 or of other tip covers configured in accordance with various embodiments of the present technology. Referring first to FIG. 2A, the tip cover 200 can include an elongated body 210 extending between a proximal end 212a and a distal end 212b. The elongated body 210 can include a wall 220 having a generally polygonal cross-section. In the illustrated embodiment, the wall 220 has a generally hexagonal cross-section having six flat edges 222 and six corners 224. The corners 224 can optionally be rounded. In other embodiments, the wall 220 can have a different generally polygonal cross-section, such as a triangular, rectangular, pentagonal, heptagonal, octagonal, nonagonal, decagonal, or another suitable cross-section. In some embodiments, the cross-section of the wall 220 is shaped as a regular polygon, having equally sized sides and equal interior angles. In some embodiments, the cross-section of the wall 220 may be a stretched or compressed polygon (e.g., a polygon extended or compressed along an axis) as opposed to a regular polygon. For example, the cross-section of the wall 220 can be a regular polygon stretched along an axis by no more than 50%, 40%, 30%, 20%, 10%, 5%, 4%, 3%, 2%, or 1% of the regular polygon.

As shown, the elongated body 210 can include a lumen, recess, or cavity 240 extending from the proximal end 212a toward the distal end 212b. In the illustrated embodiment, the cavity 240 has a circular cross-section. In other embodiments, however, the cavity 240 can have a differently-shaped cross-section. The cavity 240 can be shaped and sized to receive a distal end of a medical device or of another tool or instrument. The elongated body 210 can also include one or more side apertures, holes, or openings 230 on the wall 220. In the illustrated embodiment, the elongated body 210 includes three openings 230 on one side of the wall 220 (e.g., a front side of the tip cover 200) and three openings 230 on an opposite side of the wall 220 (e.g., a back side of the tip cover 200). While the openings 230 have hexagonal shapes and are generally similar or identical in size in the illustrated embodiment, the openings 230 can have other shapes, different sizes, etc. The openings 230 can permit venting and/or gas/liquid flow between an interior of the tip cover 200 and an exterior of the tip cover 200, and/or can allow a distal end of a medical device or component received in the cavity 240 to be sterilized while the tip cover 200 is disposed thereon.

Referring next to FIG. 2B, the elongated body 210 can have a length L1 between 15.2-25.4 mm, such as about 20.3 mm. Each of the openings 230 can have a cross-sectional dimension H1 between 1.5-2.6 mm, such as about 2.2 mm. In the illustrated embodiment, the elongated body 210 tapers toward the distal end 212b (e.g., such that a width of the tip cover 200 at the distal end 212b is smaller than a width of the tip cover 200 at the proximal end 212a). In the illustrated embodiment, the openings 230 on the two sides of the wall 220 are aligned. In other embodiments, however, the elongated body 210 can be substantially straight and/or the openings 230 on one side of the wall 220 can be misaligned with one another and/or with the opening 230 on another side of the wall 220. Referring next to FIG. 2C, the elongated body 210 does not include openings 230 on the left and right sides. In other embodiments, the elongated body 210 can include openings 230 on the left side, the right side, or both the left and right sides. In these and other embodiments, the elongated body 210 can omit openings 230 on the front and/or back sides. Referring next to FIG. 2D, the cavity 240 does not extend fully through the distal end 212b. Also, because the elongated body 210 is tapered, the openings 230 can be visible from the perspective shown in FIG. 2D.

Referring next to FIG. 2E, individual ones of the flat edges 222 of the hexagonal wall 220 at the proximal end 212a can have a dimension D1 between 1.5-2.6 mm, such as about 2.1 mm. The hexagonal wall 220 at the proximal end 212a can have a dimension D2 between opposites flat edges 222 between 3.8-5.1 mm, such as about 4.4 mm. The cavity 240 at the proximal end can have a cross-sectional dimension D3 (e.g., diameter) between 2.0-3.9 mm, such as about 2.8 mm. As shown, the cavity 240 can narrow away from the proximal end 212a (e.g., in the direction into the page).

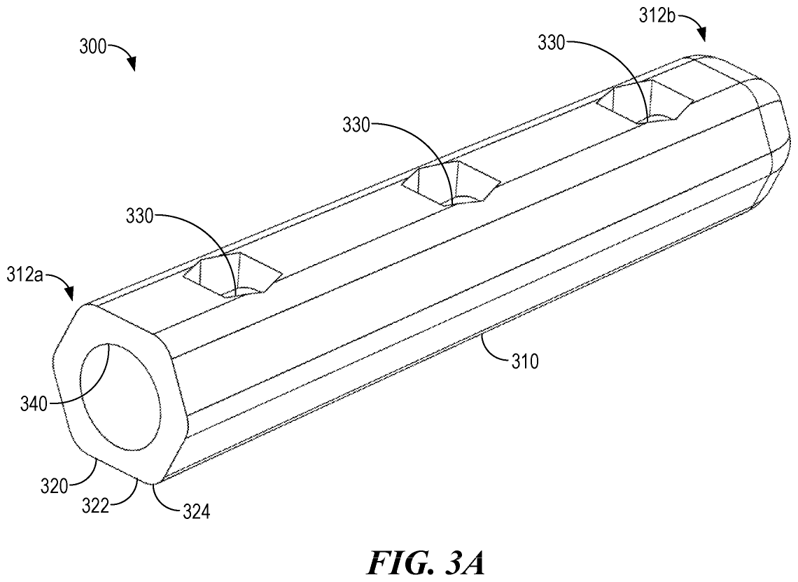

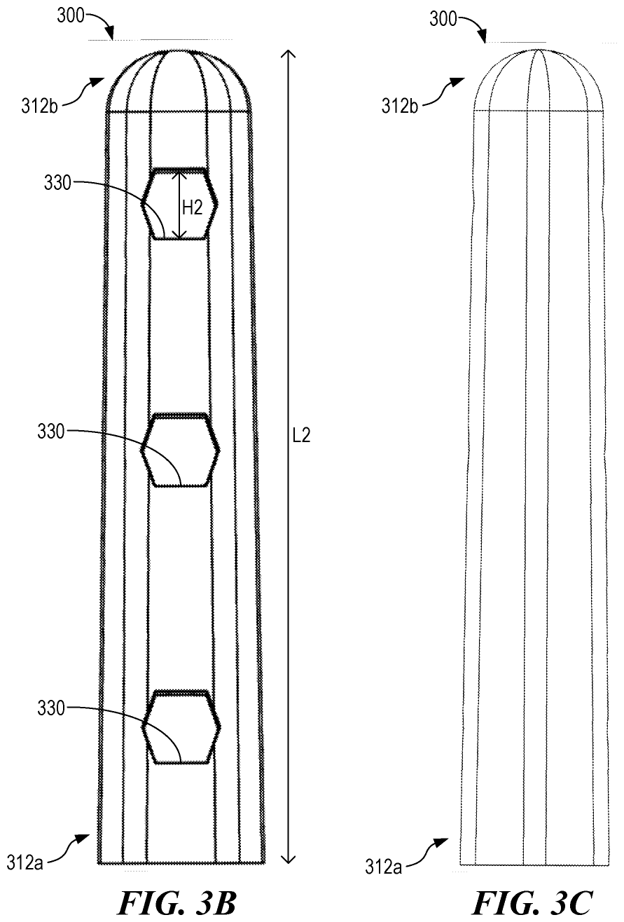

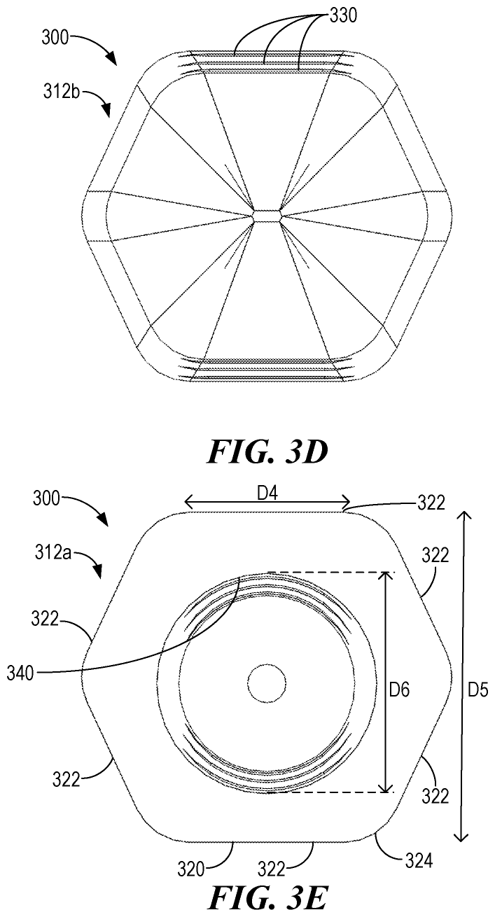

FIG. 3A-3E are perspective, front, side, top, and bottom views, respectively, of a medium-sized tip cover 300 configured in accordance with various embodiments of the present technology. The tip cover 300 can be an example of the tip covers 100 illustrated in FIG. 1 or of other tip covers configured in accordance with various embodiments of the present technology. Also, the tip cover 300 can be generally similar to the tip cover 200 of FIG. 2A-2E. For example, referring first to FIG. 3A, the tip cover 300 can include an elongated body 310 extending between a proximal end 312a and a distal end 312b. The elongated body 310 can include a wall 320 having a generally hexagonal cross-section with six flat edges 322 and six corners 324. The corners 324 can optionally be rounded. The elongated body 310 can include a cavity 340 and one or more openings 330. As discussed above with respect to the tip cover 200, the wall 320, the one or more openings 330, and the cavity 340 can have various shapes.

Referring to FIG. 3B, the elongated body 310 can have a length L2 of about 26.4 mm. Each of the openings 330 can have a cross-sectional dimension H2 of about 2.4 mm. In the illustrated embodiment, the elongated body 310 tapers toward the distal end 312b, and the openings 330 on the two sides of the wall 320 are aligned. In other embodiments, however, the elongated body 310 can be substantially straight and/or the openings 330 can be misaligned.

Referring next to FIG. 3E, individual ones of the flat edges 322 of the hexagonal wall 320 at the proximal end 312a can have a dimension D4 between 1.5-2.6 mm, such as about 2.2 mm. The hexagonal wall 220 at the proximal end 312a can have a dimension D5 between opposites flat edges 322 between 4.0-5.6 mm, such as about 4.8 mm. The cavity 340 at the proximal end can have a cross-sectional dimension D6 (e.g., diameter) between 2.5-5.1 mm, such as about 3.2 mm. As shown, the cavity 340 can narrow away from the proximal end 312a (e.g., in the direction into the page).

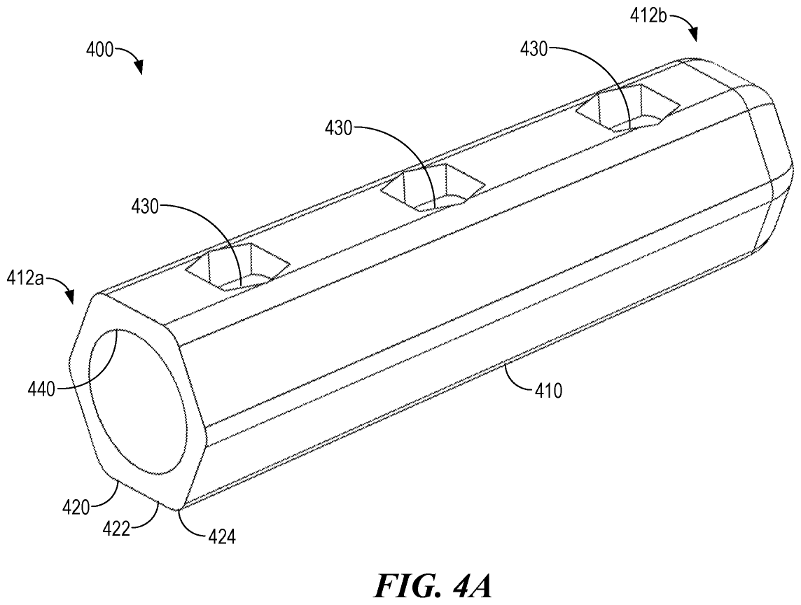

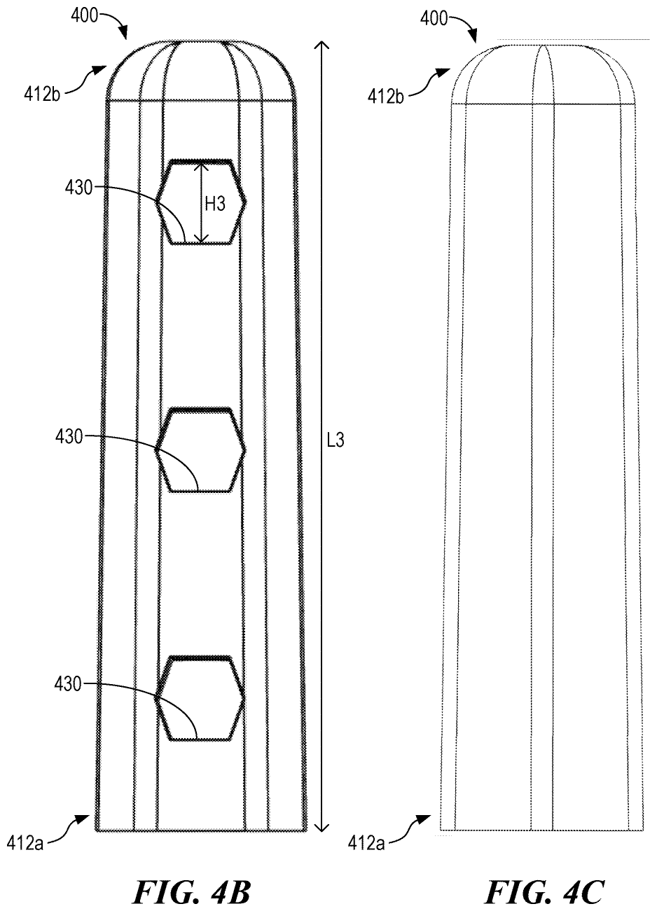

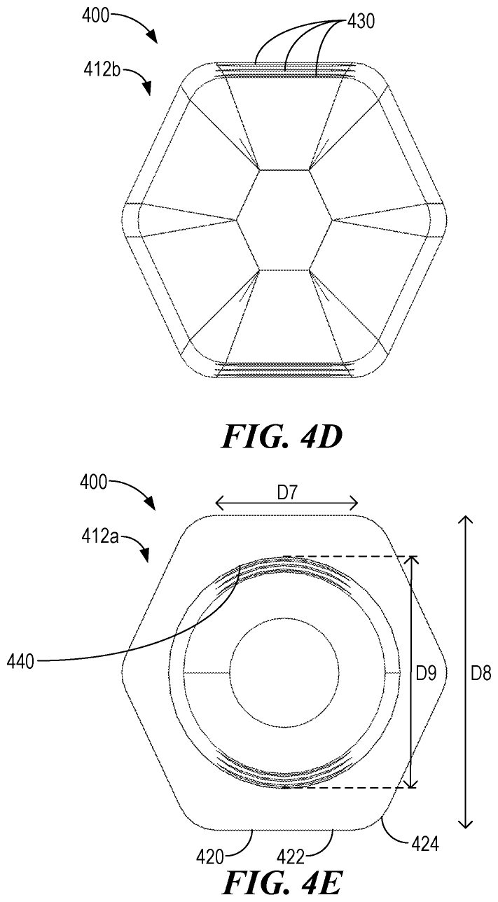

FIG. 4A-4E are perspective, front, side, top, and bottom views, respectively, of a large-sized tip cover 400 configured in accordance with various embodiments of the present technology. The tip cover 400 can be an example of the tip covers 100 illustrated in FIG. 1 or of other tip covers configured in accordance with various embodiments of the present technology. Also, the tip cover 400 can be generally similar to the tip cover 200 of FIG. 2A-2E. For example, referring first to FIG. 4A, the tip cover 400 can include an elongated body 410 extending between a proximal end 412a and a distal end 412b. The elongated body 410 can include a wall 420 having a generally hexagonal cross-section with six flat edges 422 and six corners 424. The corners 424 can optionally be rounded. The elongated body 410 can include a cavity 440 and one or more openings 430. As discussed above with respect to the tip cover 200, the wall 420, the one or more openings 430, and the cavity 440 can have various shapes.

Referring to FIG. 4B, the elongated body 410 can have a length L3 between 20.3-36.5 mm, such as about 26.4 mm. Each of the openings 430 can have a cross-sectional dimension H3 between 2.0-3.9 mm, such as about 2.8 mm. In the illustrated embodiment, the elongated body 410 tapers toward the distal end 412b, and the openings 430 on the two sides of the wall 420 are aligned. In other embodiments, however, the elongated body 410 can be substantially straight and/or the openings 430 can be misaligned.

Referring next to FIG. 4E, individual ones of the flat edges 422 of the hexagonal wall 420 at the proximal end 412a can have a dimension D7 between 2.0-3.9 mm, such as about 2.9 mm. The hexagonal wall 220 at the proximal end 412a can have a dimension D8 between opposites flat edges 422 between 5.0-7.7 mm, such as about 6.8 mm. The cavity 440 at the proximal end can have a cross-sectional dimension D9 (e.g., diameter) between 4.0-6.4 mm, such as about 5.0 mm. As shown, the cavity 440 can narrow away from the proximal end 412a (e.g., in the direction into the page).

Referring to FIG. 2A-4E together, the small-sized tip cover 200, the medium-sized tip cover 300, and the large-sized tip cover 400 can have different dimensions to accommodate different medical instruments. In particular, the cross-sectional dimensions D3, D6, D9 of the cavities 240, 340, 440 can be sized differently to receive differently-sized distal ends or tips of medical devices. As indicated above, other dimensions of the tip covers 200, 300, 400 can be similar or different. It is appreciated that the dimensions provided above are merely examples, and that tip covers of the present technology can have other suitable dimensions. For example, other tip covers of the present technology can include a cavity having a cross-sectional dimension (e.g., diameter) of up to 10 mm, 12 mm, 14 mm, 16 mm, 18 mm, 20 mm, or more to accommodate even larger medical device or other components.

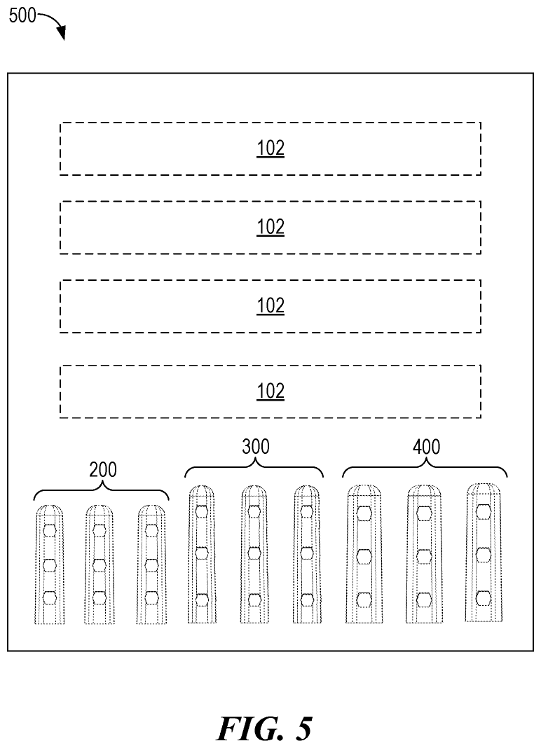

FIG. 5 is a schematic plan view of a surgical or medical kit 500 (“the kit 500”) configured in accordance with various embodiments of the present technology. As shown, the kit 500 can include one or more of each of the small-sized tip covers 200, the medium-sized tip covers 300, and/or the large-sized tip covers 400. Accordingly, depending on the particular medical device to be sanitized, a technician (or other user) can select the appropriately sized tip cover from among the tip covers 200, 300, 400 included in the kit 500. In some embodiments, the kit 500 can optionally include one or more medical devices 102 (drawn schematically in phantom lines).

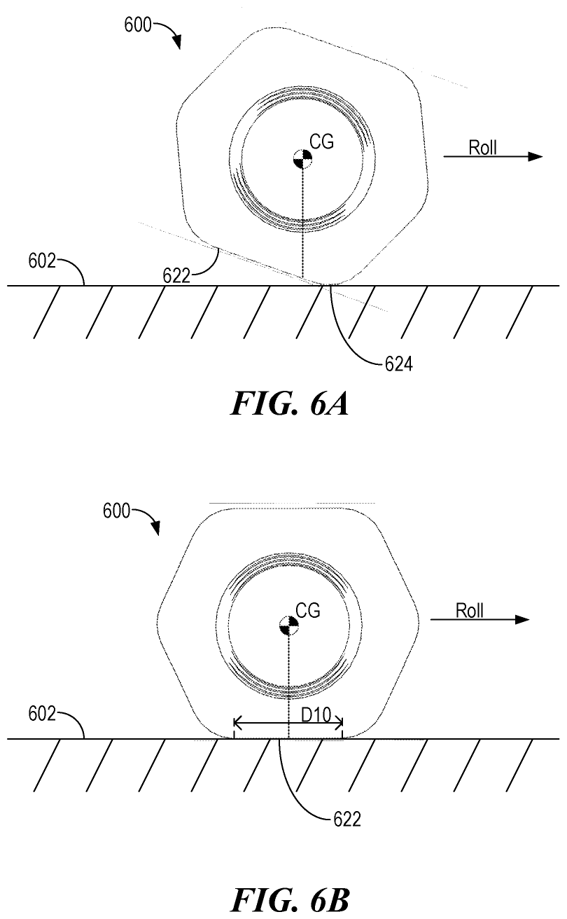

FIGS. 6A and 6B illustrate anti-rolling and anti-rocking features of a tip cover 600 configured in accordance with various embodiments of the present technology. The tip cover 600 can be an example of any one of the tip covers 100, 200, 300, 400 illustrated and described herein, or of other tip covers configured in accordance with various embodiments of the present technology. The tip cover 600 can have a generally polygonal (e.g., hexagonal) cross-section. When the tip cover 600 (which may or may not be currently disposed on a distal end of a medical device) is at rest on a surface 602 (FIG. 6B), a flat side 622 of the tip cover 600 can be in contact with the surface 602. The flat side 622 is defined as the portion that contacts the surface 602 when at rest (e.g., excluding the corners 624). Because the cross-section of the tip cover 600 is generally polygonal, the tip cover 600 is most likely to be at rest on one of its flat sides 622. The static friction between the flat side 622 and the surface 602 can help resist lateral movement of the tip cover 600.

In a healthcare setting, the tip cover 600 (which again may or may not be disposed on a distal end of a medical device) may be dropped, pushed, displaced, or otherwise moved such that the tip cover 600 rolls along the surface 602 in the direction of the arrow labeled “Roll” (FIG. 6A). The tip cover 600 can continue to roll until the tip cover 600 loses momentum. The moment of inertia of the tip cover 600 increases with its cross-sectional dimension, so a larger tip cover 600 can resist and cease rolling more easily than a smaller tip cover 600. The size of the tip cover 600, however, may be dictated by the size of distal ends of medical devices on or over which the tip cover 600 is configured to be disposed.

Also, as a general principle, the closer the cross-section of the tip cover 600 approximates a perfect circle, the harder it will be for the tip cover 600 to stop rolling. Therefore, it is expected that a tip cover 600 having a polygonal cross-section with fewer sides will better resist rolling. The polygonal cross-sectional shape of the tip cover 600, however, may also be selected based at least in part on ergonomics, manufacturability, and overall design fit for the medical device 102. For example, the illustrated hexagonal cross-section may be easier to handle and pick up than a triangular cross-section. As another example, polygons with fewer sides inherently have sharper corners, so tip covers having polygonal cross-sections with fewer sides may be more difficult to manufacture, more likely to puncture a sterilization pouch, and/or more prone to wear and tear compared to tip covers having polygonal cross-sections with more sides.

Once the rolling tip cover 600 loses momentum, if the tip cover 600 is on one of the corners 624, the tip cover 600 will most likely drop onto the flat side 622 that is underneath the center of gravity (CG) of the tip cover 600. As the tip cover 600 rotates onto that flat side 622, the tip cover 600 may gain a small amount of momentum that may cause the tip cover 600 to continue rotating beyond the particular flat side 622. The flat side 622, however, can provide enough surface area to absorb such momentum upon impact with the surface 602, thereby preventing or at least resisting or minimizing rocking motions. Also, due to the generally polygonal cross-sectional shape, a tip cover 600 having an n-degree polygonal cross-section has n lines of symmetry, and the CG can be at the intersection of the n lines of symmetry. Accordingly, regardless of which particular flat side 622 the tip cover 600 lands on, the CG can be positioned above and generally centered with the particular flat side 622, providing stability that resists rocking motions, as shown in FIG. 6B. In some embodiments, when the tip cover 600 is at rest and regardless of which flat side 622 the tip cover 600 rests on, the CG can be positioned no more than 30%, 20%, 10%, 5%, 4%, 3%, 2%, or 1% of the length D10 of the flat side 622 laterally away from the center of the flat side 622.

Referring to FIG. 1-6B together, the generally polygonal cross-sections of the tip covers of the present technology can provide multiple flat sides, multiple lines of symmetry, and a center of gravity that altogether provide a high degree of stability. Accordingly, tip covers of the present technology, regardless of the specific shape or size, can resist rolling and rocking motions on a surface to a higher degree compared to many existing tip covers. This is expected to be the case both (a) when the tip covers are disposed on or over tips of medical devices, tool, or other instruments, and (b) when the tip covers are not disposed on or over such tips (e.g., when the tip covers are not in use). The anti-rolling and anti-rocking features of the tip covers described herein are therefore expected to provide several advantages. For example, when tip covers of the present technology drop onto a surface (e.g., a table, a tray) or the floor (e.g., due to mishandling by a technician, accidental shaking of a table on which the medical devices are placed), the tip covers can prevent or at least resist rolling and thereby quickly come to a stop. Thus, the tip covers can (i) reduce the likelihood of the tip covers from getting lost (e.g., by rolling underneath a cabinet), (ii) provide a convenience factor to personal by reducing the effort and time it takes to pick the tip covers back up, (iii) reduce the risk of contaminating (e.g., via coming into contact with) other tip covers or other medical devices, components, tools, or material in the technician's work area, and more. Additionally, or alternatively, when the tip covers of the present technology are at rest on a surface (e.g., a table or tray), the tip covers can resist rocking or other lateral movement. In turn, it is expected that tip covers of the present technology will, when disposed on tips of medical devices/tool, and/or other instruments, better maintain these objects in place and/or better limit lateral movement of such objects, especially in comparison to existing tip covers.

### C. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. The terms “about,” “approximately,” and “substantially” as used herein shall be interpreted to mean within ±10% of the stated value. Additionally, all ranges disclosed herein are to be understood to encompass the endpoints, and any and all subranges subsumed therein. For example, a range of “1 to 10” includes any and all subranges between (and including) the minimum value of 1 and the maximum value of 10 (e.g., any and all subranges having a minimum value of equal to or greater than 1 and a maximum value of equal to or less than 10, such as 5.5 to 10).

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

What is claimed is:

1. A tip cover for covering a distal end of a medical device, the tip cover comprising:
an elongated body extending between a proximal end and a distal end thereof, the elongated body including:
a wall having a cross-section shaped generally as an n-degree polygon such that the wall has n flat sides; and
a cavity surrounded by the wall and extending from the proximal end of the elongated body, wherein the cavity is configured to receive the distal end of the medical device therein,
wherein, when the elongated body is at rest on a surface on any one of the n flat sides, a center of gravity of the elongated body is positioned no more than 20% of a length of the one of the n flat sides laterally away from a center of the one of the n flat sides.

2. The tip cover of claim 1, wherein, when the elongated body is at rest on the surface on the one of the n flat sides, the center of gravity of the elongated body is positioned no more than 10% of the length of the one of the n flat sides laterally away from the center of the one of the n flat sides.

3. The tip cover of claim 1, wherein the cross-section of the wall is shaped generally as a hexagon having six flat sides and six corners.

4. The tip cover of claim 3, wherein the six corners are rounded.

5. The tip cover of claim 1, wherein the cross-section of the wall is shaped generally as a triangle, a square, a pentagon, a heptagon, an octagon, a nonagon, or a decagon.

6. The tip cover of claim 1, wherein the cross-section of the wall is shaped as a regular n degree polygon.

7. The tip cover of claim 1, wherein the cross-section of the wall is shaped as a stretched n-degree polygon, wherein the stretched n-degree polygon is stretched along an axis by no more than 20% of a corresponding regular n-degree polygon.

8. The tip cover of claim 1, wherein the elongated body is tapered towards the distal end thereof such that a width of the tip cover at the distal end is smaller than a width of the tip cover at the proximal end.

9. The tip cover of claim 1, wherein the elongated body includes a plurality of openings configured to permit gas or liquid flow between an interior of the tip cover and an exterior of the tip cover.

10. The tip cover of claim 9, wherein the plurality of openings are configured to allow the distal end of the medical device to be sterilized while covered by the tip cover.

11. The tip cover of claim 1, wherein the cavity has a circular cross-section.

12. The tip cover of claim 1, wherein the cavity narrows away from the proximal end toward the distal end.

13. The tip cover of claim 1, wherein the tip cover is formed at least in part of a thermoplastic elastomer, a thermoplastic polyurethane, a silicone, and/or a bio-resin.

14. A medical kit, comprising:
a plurality of tip covers including one or more tip covers of a first size and one or more tip covers of a second size, wherein each of the plurality of tip covers includes:
an elongated body extending between a proximal end and a distal end thereof, the elongated body including:
a wall having a cross-section shaped generally as an n-degree polygon such that the wall has n flat sides; and
a cavity surrounded by the wall and extending from the proximal end of the elongated body,
wherein, when the tip cover is at rest on a surface on any one of the n flat sides, a center of gravity of the tip cover is positioned no more than 20% of a length of the one of the n flat sides laterally away from a center of the one of the n flat sides,
wherein the cavities of the tip covers of the first size are sized differently from the cavities of the tip covers of the second size such that the tip covers of the first and second sizes are configured to receive differently sized distal ends of instruments.

15. The medical kit of claim 14, wherein:
the cavities of the tip covers of the first size have first cross-sectional dimensions that fall within a first dimension range;
the cavities of the tip covers of the second size have second cross-sectional dimensions that fall within a second dimension range different from the first dimension range; and
the first and second dimension ranges correspond to different ones of the following dimension ranges: 2.0-3.8 mm, 2.5-5.1 mm, and 4.1-6.4 mm.

16. The medical kit of claim 15, wherein:
the plurality of tip covers further includes one or more tip covers of a third size;
the cavities of the tip covers of the third size are sized differently from the cavities of the tip covers of the first and second sizes such that the tip covers of the first, second, and third sizes are configured to receive differently sized distal ends of instruments;
the cavities of the tip covers of the third size have third cross-sectional dimensions that fall within a third dimension range different from the first and second dimension ranges; and
the first, second, and third dimension ranges correspond to different ones of the following dimension ranges: 2.0-3.8 mm, 2.5-5.1 mm, and 4.1-6.4 mm.

17. The medical kit of claim 14, further comprising one or more instruments, each instrument having a distal end sized to fit in the cavity of at least one of the plurality of tip covers.

18. An anti-roll and anti-rocking tip cover, comprising:
an elongated body extending between a proximal end and a distal end thereof, the elongated body including:
a wall having a cross-section shaped generally as a hexagon such that the wall has six flat sides; and
a cavity surrounded by the wall and extending from the proximal end of the elongated body toward the distal end, wherein the cavity is configured to receive a distal end of a medical device therein.

19. The anti-roll and anti-rocking tip cover of claim 18, wherein, when the elongated body is at rest on a surface on any one of the six flat sides, a center of gravity of the elongated body is positioned no more than 20% of a length of the one of the six flat sides laterally away from a center of the one of the six flat sides.

20. The anti-roll and anti-rocking tip cover of claim 18, wherein the elongated body further includes one or more openings on the wall configured to permit gas or liquid flow between an interior of the tip cover and an exterior of the tip cover.
